(19) United States
(12) Patent Application Publication
(10) Pub. No.: US 2025/0241719 A1
(43) Pub. Date: Jul. 31, 2025

(54) ADAPTER FOR MANUAL ACTUATION OF SURGICAL INSTRUMENT

(71) Applicant: Intuitive Surgical Operations, Inc., Sunnyvale, CA (US)

(72) Inventors: Scott E. MANZO, Shelton, CT (US); Adam J. ROSS, Prospect, CT (US); James FERRO, Shelton, CT (US); Craig R. GERBI, Half Moon Bay, CA (US)

(73) Assignee: Intuitive Surgical Operations, Inc., Sunnyvale, CA (US)

(21) Appl. No.: 19/100,030

(22) PCT Filed: Jul. 25, 2023
(86) PCT No.: PCT/US2023/028548

§ 371 Date: Jan. 30, 2025

Related U.S. Application Data
(60) Provisional application No. 63/394,076, filed on Aug. 1, 2022.

(51) Int. Cl. A61B 34/00; A61B 18/00; A61B 18/08; A61B 34/30; A61B 34/35

(52) U.S. Cl. CPC A61B 34/71; A61B 18/085; A61B 34/35; A61B 2018/00172; A61B 2018/00184; A61B 2018/00601; A61B 2034/305

(57) ABSTRACT

A manual drive adapter for a robotic instrument includes an adapter housing configured to be releasably coupled to a force transmission mechanism of the robotic instrument. The force transmission mechanism includes a drive member interface and an input drive member configured to engage with a robotic drive output of a teleoperated surgical system. The adapter includes an output drive interface that includes an output drive member and is coupled to the adapter housing. The output drive interface is configured to be releasably coupled to the drive member interface of the robotic instrument such that the output drive member is engageable with the input drive member of the robotic instrument. A manual actuator of the adapter is operably coupled to the output drive member such that movement of the manual actuator by a user causes the output drive member to operate one or more degrees of freedom of the robotic instrument.

### CROSS-REFERENCE TO RELATED APPLICATIONS

This application claims benefit of priority to U.S. Provisional Application Ser. No. 63/394,076, entitled “Adapter for Manual Actuation of Surgical Instrument,” filed Aug. 1, 2022, which is incorporated herein by reference in its entirety.

### BACKGROUND

The embodiments described herein relate to medical devices, and more specifically to robotic instruments. More particularly, the embodiments described herein relate to medical devices that include a manual drive adapter that can be removably coupled to a robotic instrument to provide for manual actuation of the robotic instrument.

During some known medical procedures, surgeons may use a robotic instrument operably coupled to a teleoperated system to perform various medical procedures. In some cases, it may be desirable for the surgeon to perform some portion of the procedure using a manually operated medical device (e.g., a device, such as a hand-held surgical instrument, in which at least a portion of a force produced by a physical input from the user, such as squeezing a trigger, rotating a knob, or depressing an actuator, is transferred via a mechanical means to an output of the device). For example, the surgeon may desire to use one or more manual instruments in conjunction with a robotic procedure, wherein the manual instruments are used concurrently with robotic instruments and/or wherein the robotic instruments are used for one portion of a procedure and manual instruments are used for another portion of the procedure. For example, the surgeon may wish to open or close the jaws of a non-robotic, manually actuated energy or non-energy surgical tool (e.g., a scissor, grasper, vessel sealer, bipolar, monopolar, harmonic, or other tool) to perform aspects of the procedure manually without use or coupling to the teleoperated system or to perform aspects that cannot be readily performed using the existing robotic instrument controlled by a teleoperated system. This requires that the surgeon use two or more separate instruments to perform a particular medical procedure, one robotic instrument controlled by the teleoperated system and a second, manual instrument that is actuated by hand. Alternatively, in some medical procedures the surgeon may desire to transition the use of an instrument from teleoperated control to manually actuated control.

Although some known robotic devices include an emergency release mechanism to allow a user to manually override the teleoperated control, such release systems are generally used only for simple operation of the device (e.g., opening the jaws) and do not allow for use of the device to perform a procedure. Such known devices do not allow the surgeon to hold and actuate the robotic instrument manually but disconnected from the teleoperated system, with the same robotic instrument being used in a teleoperated manner at other times during the procedure.

Thus, a need exists for an adapter to provide for easily converting a robotic tool between use with a teleoperated system, in which the robotic tool is operated by controls of the teleoperated system, and use with a manual actuator, in which the robotic tool is not controlled by the teleoperated system, but rather is controlled by manual inputs provided by the surgeon.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a manual drive adapter for a robotic instrument includes an adapter housing configured to be releasably coupled to a force transmission mechanism of the robotic instrument. The force transmission mechanism of the robotic instrument includes a drive member interface and an input drive member configured to engage with a robotic drive output of a teleoperated surgical system. An output drive interface includes an output drive member and is coupled to the adapter housing. The output drive interface is configured to be releasably coupled to the drive member interface of the robotic instrument such that the output drive member is engageable with the input drive member of the robotic instrument. A manual actuator is operably coupled to the output drive member such that movement of the manual actuator by a user causes the output drive member to operate one or more degrees of freedom of the robotic instrument. In some embodiments, when the output drive interface is coupled to the drive member interface of the robotic instrument, the input drive member of the robotic instrument is prevented from being engaged with a robotic drive output of a teleoperated surgical system.

In some embodiments, the robotic instrument includes a shaft having a proximal end portion coupled to the force transmission mechanism of the robotic instrument and a distal end portion coupled to an end effector, where movement of the manual actuator by the user causes the output drive member to move the input drive member of the robotic instrument to actuate the end effector. In some embodiments, the end effector includes a gripping tool, and movement of the manual actuator actuates the gripping tool when the output drive member is coupled to the input drive member of the robotic instrument. In some embodiments, the end effector includes a cutting tool, and movement of the manual actuator actuates the cutting tool to move relative to jaws of the end effector (i.e., proximally or distally) when the output drive member is coupled to the input drive member of the robotic instrument.

In some embodiments, the output drive interface includes multiple output drive members configured to be engaged with multiple input drive members of the robotic instrument. In some such embodiments, the input drive member is a first input drive member of the multiple input drive members, the manual actuator is a first manual actuator, and the output drive member is a first output drive member of the multiple output drive members, and the manual drive adapter further includes a second manual actuator. The second manual actuator is operably coupled to a second output drive member of the multiple output drive members, and the second manual actuator can be manually actuated by the user to cause the second output drive member to move a second input drive member of the multiple input drive members of the robotic instrument when the adapter housing is coupled to the force transmission mechanism of the robotic instrument.

In some embodiments having a second manual actuator, the robotic instrument includes a shaft having a proximal end portion coupled to the force transmission mechanism of the robotic instrument and a distal end portion coupled to an end effector that includes a gripping tool and a cutting tool. In such an embodiment, the first manual actuator is configured to manually actuate the gripping tool when the first output drive member is coupled to the first input drive member of the robotic instrument and the second manual actuator is configured to manually actuate the cutting tool to move relative to jaws of the end effector when the second output drive member is coupled to the second input drive member of the robotic instrument.

In some embodiments, the robotic instrument includes a shaft having a proximal end portion coupled to the force transmission mechanism of the robotic instrument and a distal end portion coupled to a wrist mechanism. The wrist mechanism is configured to rotate about a rotation axis to move a position of the wrist mechanism about the rotation axis, and the first manual actuator is configured to manually actuate the tool when the first output drive member is coupled to the first input drive member of the robotic instrument. The manual drive adapter includes a second manual actuator configured to cause the wrist mechanism to be locked in a fixed position relative to the shaft.

In some embodiments having a first a manual actuator and a second manual actuator, the distal end portion of the shaft is coupled to a link, the link is coupled to the gripping tool and the cutting tool, and the link is configured to rotate about a rotation axis to move a position of at least one of the gripping tool or the cutting tool about the rotation axis. In some such embodiments, the manual drive adapter further includes a third manual actuator configured to be manually actuated by the user to lock the link in a fixed position relative to the shaft and prevent at least one of the gripping tool or the cutting tool from moving about the rotation axis.

In some embodiments, the manual drive adapter further includes a lockout mechanism that prevents the second manual actuator from being actuated unless the first manual actuator is actuated. In some embodiments, the manual drive adapter includes a torque limiter coupled to the manual actuator and configured to limit a torque applied to the drive member when the manual actuator is moved by the user. In some embodiments, the torque limiter includes a torsion spring. In some embodiments, a power connection is configured such that when the adapter housing is coupled to the force transmission mechanism of the robotic instrument, the robotic instrument is electrically coupled to a power source. In some embodiments, the manual drive adapter is provided within a sterile kit and configured for a single use.

In some embodiments, a manual drive adapter includes an adapter housing configured to be releasably coupled to a housing of a robotic instrument. The force transmission mechanism of the robotic instrument includes a first input drive member and a second input drive member. The first input drive member is configured to engage with a first robotic drive output of a teleoperated surgical system, and the second input drive member is configured to engage with a second robotic drive output of the teleoperated surgical system. A first manual actuator is coupled to the adapter housing and is operably coupled to the first input drive member such that movement of the first manual actuator causes the first input drive member to operate a first tool of the robotic instrument. A second manual actuator is coupled to the adapter housing and is operably coupled to the second input drive member such that movement of the second manual actuator causes the second input drive member to operate a second tool of the surgical instrument.

In some embodiments, the robotic instrument includes a shaft having a proximal end portion coupled to the force transmission mechanism of the robotic instrument and a distal end portion coupled to a link. The link is coupled to the first tool and configured to rotate about a rotation axis to move a position of the first tool about the rotation axis. In some such embodiments, the manual drive adapter further includes a third actuator configured to be manually actuated to lock the link in a fixed position relative to the shaft and prevent the first tool from moving about the rotation axis.

In some embodiments, the first tool is a gripping tool and the second tool is a cutting tool. When the adapter housing is coupled to the force transmission mechanism of the robotic instrument, movement of the first manual actuator actuates the gripping tool and movement of the second manual actuator actuates the cutting tool to move relative to jaws of the gripping tool.

Other medical instruments, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control console of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5A is an illustration of a portion of the teleoperated system of FIG. 1, illustrating an instrument carriage of the manipulator unit, according to an embodiment.

FIG. 5B is an illustration of the instrument carriage of FIG. 5A shown coupled to a sterile adapter of the manipulator unit and a robotic instrument coupled thereto.

FIG. 5C is an exploded view of the instrument carriage, sterile adapter and robotic instrument of FIG. 5B.

FIG. 6A is a schematic illustration of a robotic instrument coupled to a teleoperated system, according to an embodiment.

FIG. 6B is a schematic illustration of the robotic instrument of FIG. 6A shown disconnected from the teleoperated system.

FIG. 6C is a schematic illustration of the robotic instrument of FIG. 6B shown coupled to a manual drive adapter, according to an embodiment.

FIG. 7 is a schematic illustration of manual drive adapter, according to an embodiment, shown coupled to a robotic instrument according to an embodiment.

FIG. 8A illustrates a manual drive adapter according to an embodiment, shown coupled to a robotic instrument.

FIG. 8B illustrates the manual drive adapter of FIG. 8A, shown decoupled from the robotic instrument.

FIG. 9 is a side view of a robotic instrument and a manual drive adapter, according to an embodiment.

FIG. 10 is a perspective distal view of a portion of the robotic instrument and manual drive adapter of FIG. 9.

FIG. 11 is a perspective view of the robotic instrument of FIG. 9 illustrating a distal view of the drive member interface of the robotic instrument.

FIG. 12A is an enlarged perspective view of a portion of the robotic instrument of FIG. 11.

FIG. 12B is an enlarged perspective view of a portion of the robotic instrument of FIG. 12A showing coupling portions of the drive member interface.

FIG. 13A is a perspective view of a distal end portion of the robotic instrument of FIG. 9.

FIG. 13B is a perspective view of a portion of the end effector of the robotic instrument of FIG. 9, with a portion of the tool member removed for illustration purposes.

FIG. 13C is a partially exploded view of a portion of the end effector of the robotic instrument of FIG. 9.

FIG. 14 is a side view of the manual drive adapter of FIG. 9, illustrating a first manual actuator in an unactuated position.

FIG. 15 is side view of the manual drive adapter of FIG. 14, illustrating the first manual actuator in an actuated position.

FIG. 16 is a perspective view of the manual drive actuator of FIG. 9 illustrating a proximal view of the output drive interface of the manual drive adapter.

FIG. 17 is a top view of the manual drive adapter of FIG. 9.

FIG. 18 is a proximal view of the manual drive adapter of FIG. 9, illustrating a proximal view of the output drive interface.

FIG. 19 is a distal perspective view of the manual drive adapter of FIG. 9.

FIG. 20 is a distal perspective view of a portion of the manual drive actuator of FIG. 9 with select components removed for illustration purposes.

FIG. 21 is a distal perspective view of a portion of the manual drive actuator of FIG. 9 with select components removed for illustration purposes.

FIG. 22 is a distal perspective view of a portion of the manual drive actuator of FIG. 9 with select components removed for illustration purposes.

FIG. 23 is a distal perspective view of a portion of the manual drive actuator of FIG. 9 with select components removed for illustration purposes.

FIG. 24A is a perspective view of a portion of the manual drive adapter of FIG. 9 illustrating the second manual actuator in a ready position.

FIG. 24B is a perspective view of a portion of the manual drive adapter of FIG. 9 illustrating the second manual actuator in an actuated position.

### DETAILED DESCRIPTION

The embodiments described herein can advantageously be used in a wide variety of grasping, cutting, and manipulating operations associated with minimally invasive surgery. In some embodiments, an end effector of a medical device can move with reference to a main body of the device in at least three mechanical degrees of freedom (DOFs), e.g., pitch, yaw, and roll (shaft roll). In some embodiments, there may also be one or more mechanical DOFs in the end effector itself, e.g., two jaws, each rotating with reference to a clevis (2 DOFs) and a distal clevis that rotates with reference to a proximal clevis (one DOF). In some embodiments, a medical device may have, for example, 5 DOF. For example, in some embodiments, a medical device can have one DOF for shaft roll, one DOF for pitch and one DOF for yaw (e.g., in the wrist), one DOF for movement of a gripping tool and one DOF for movement of a knife. In some embodiments, a medical device can include an end effector that has a gripper with 2 DOF and a knife that provides 1 DOF. However, in various embodiments, medical devices may have more or fewer DOFs.

In some embodiments, the medical devices of the present application enable motion of a robotic instrument (also referred to as medical instrument) in at least three degrees of freedom (e.g., about a pitch axis, a yaw axis, and a shaft axis). Moreover, the robotic instruments described herein include one or more actuation elements (such as cables) that can be secured to a force transmission mechanism of the instrument and secured to an end effector at a distal end of the robotic instrument. In some embodiments, the force transmission mechanism may be at a proximal end of the instrument, but the disclosure is not limited thereto or thereby, and in other embodiments, the force transmission mechanism may be positioned at an intermediate portion of the instrument between proximal and distal ends of the instrument. As described herein, the robotic instrument can be used within a teleoperated system and the cables can be driven by one or more actuators (e.g., electric motors) to move an end effector of the robotic instrument.

During some medical procedures, surgeons may use a robotic instrument operably coupled to a teleoperated system to perform various medical procedures. In some cases, it may be desirable for the surgeon to perform some portion of the procedure using a manually operated medical device. For example, the surgeon may wish to open a non-robotic, manually actuated energy or non-energy surgical tool (e.g., a scissor, grasper, vessel sealer, bipolar, monopolar, harmonic, or other tool) to perform aspects of the procedure manually without use of the teleoperated system or to perform aspects that cannot be readily performed using existing robotic tools controlled by a teleoperated system. In certain situations, the surgeon may use two or more separate tools to perform a particular medical procedure.

As described herein, manual drive adapters are provided for releasably attaching to a robotic instrument to hold and actuate the robotic instrument manually. The robotic instrument can be coupled to and operated by a teleoperated system, and the instrument may be detached from the teleoperated system and coupled to the manual drive adapter to perform a portion of a medical procedure. This allows the surgeon to use the same robotic instrument during both a robotic operated part of a medical procedure and a manual operated part of the procedure. The manual drive adapters can eliminate the time and expense of using a separate new tool, which can provide greater flexibility to the operator and reduce the overall cost of a procedure by eliminating the need to use an additional tool. Such an adapter can also provide consistency within the procedure by allowing the surgeon to use a familiar tool with expected and/or preferred performance within the procedure. In some embodiments, the manual drive adapters are provided in a sterile kit and provided for a single use.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a” “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of the invention are described primarily in terms of an implementation using a da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4200, Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

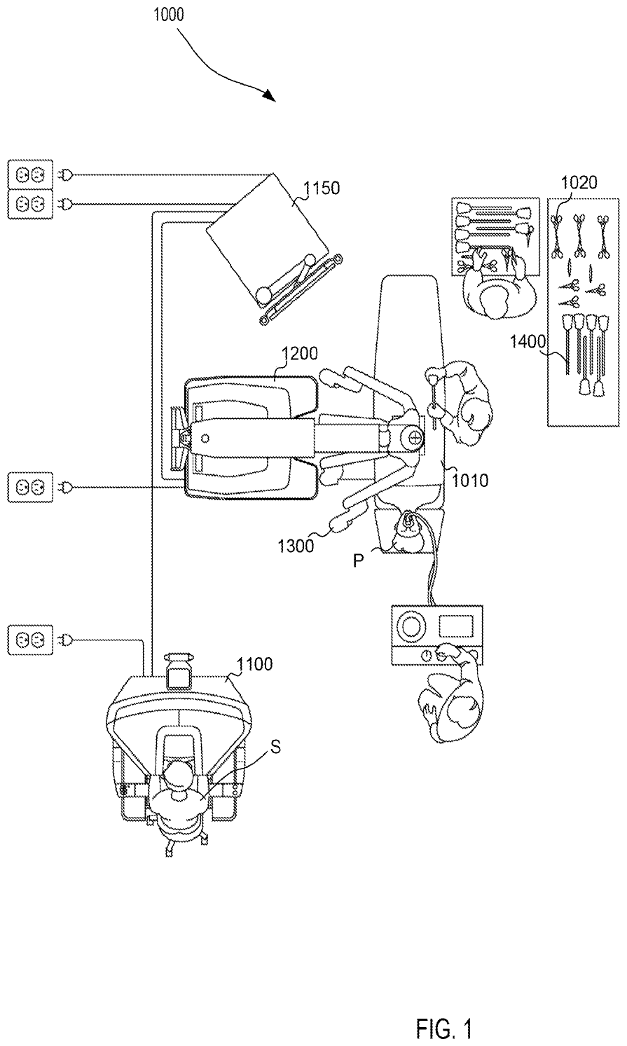

FIG. 1 is a plan view illustration of a teleoperated surgical system 1000 that operates with at least partial computer assistance (a “telesurgical system”). Both telesurgical system 1000 and its components are considered medical devices. Teleoperated system 1000 is a Minimally Invasive Robotic Surgical (MIRS) system used for performing a minimally invasive diagnostic or surgical procedure on a Patient P who is lying on an Operating table 1010. The system 1000 can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument 1400 removably coupled to the arm assembly 1300. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

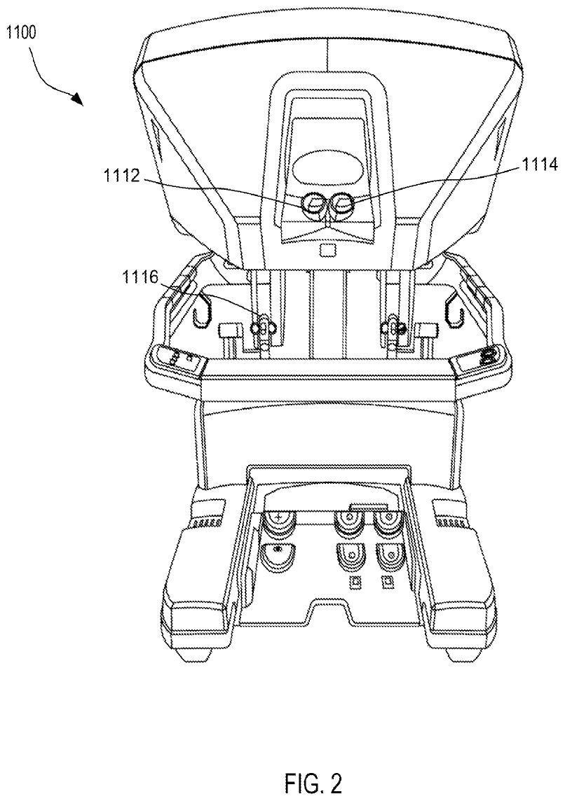

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

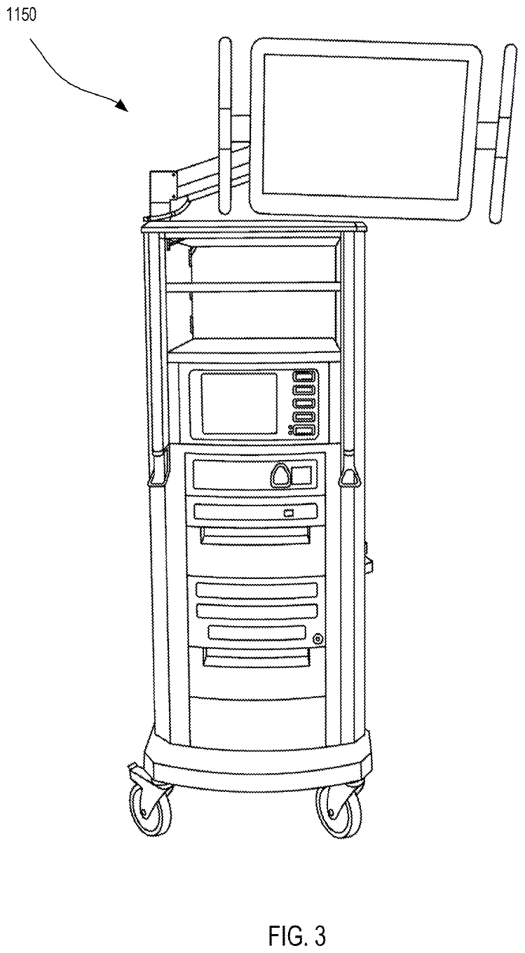

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

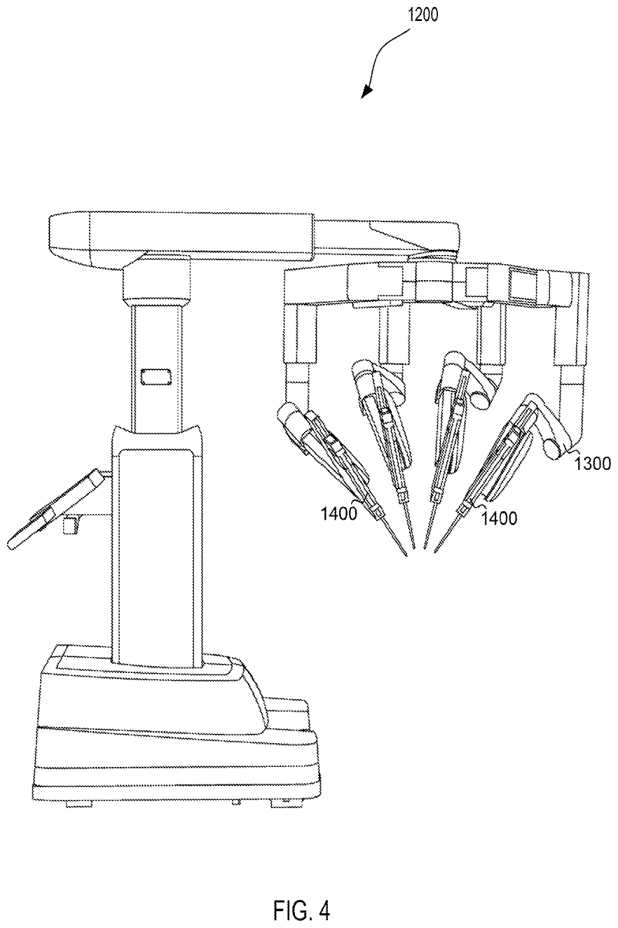

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

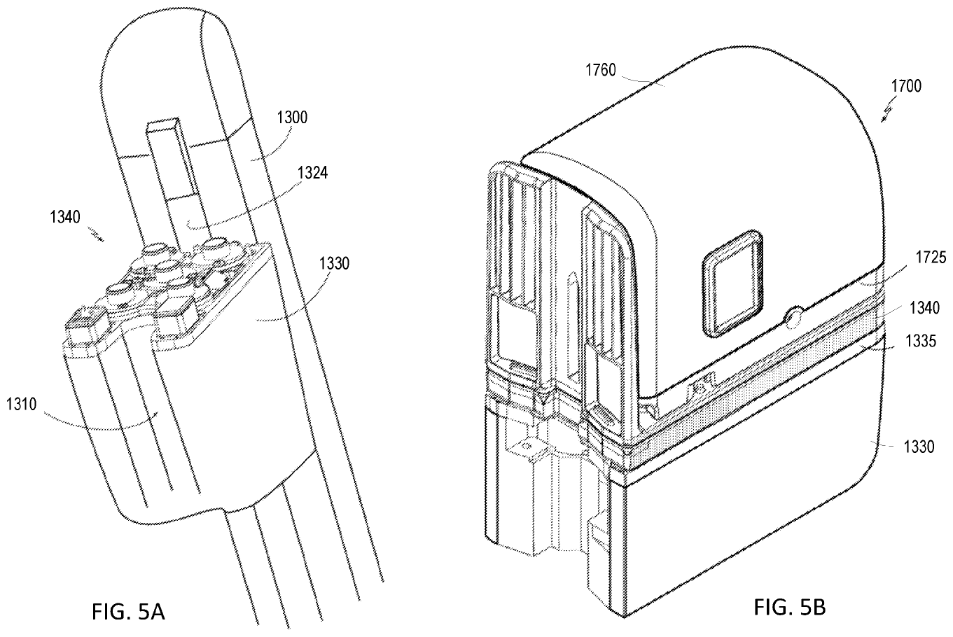

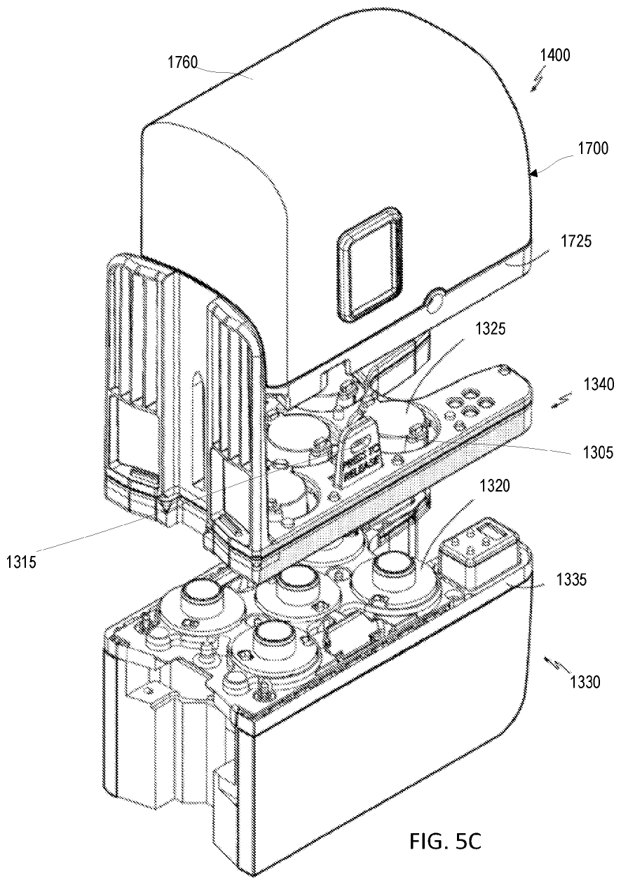

FIG. 5A is a perspective view of a portion of an arm assembly 1300 and an instrument carriage 1330 to which an instrument 1400 can be removably coupled. The instrument carriage 1330 includes teleoperated actuators to provide controller motions to the instrument 1400, which translates into a variety of movements of a tool or tools at a distal end of the instrument 1400. The arm assembly 1300 includes a connecting portion 1324 in which the instrument carriage 1330 can be coupled. The instrument carriage 1330 may be translatable relative to the arm assembly 1300, for example, along an insertion axis extending between a proximal end and a distal end of the arm-n assembly 1300 for insertion and removal of the instrument into a patent. In addition, the arm assembly 1300 can provide additional degrees of freedom to orient and position the instrument carriage 1330 and instrument 1400 at a desired location. When an instrument 1400 is coupled to the instrument carriage 1330, input provided by a surgeon to the user control unit 1100 (a “master” command) is translated into a corresponding action by the surgical instrument 1400 (a “slave” response) via robotic drive outputs 1320 of the instrument carriage 1330 that are operatively coupled to inputs on the instrument 1400 (see FIG. 5C).

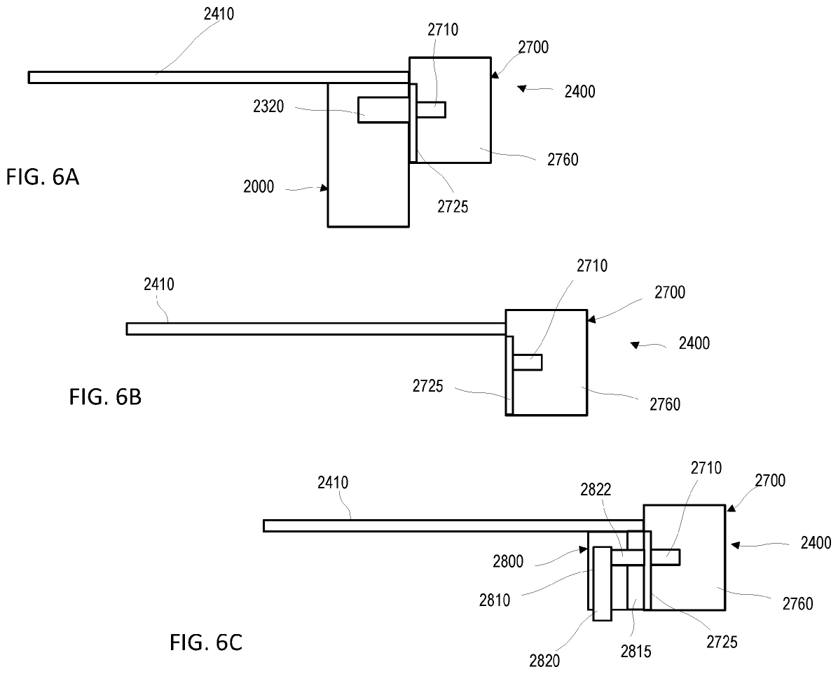

Surgical instruments 1400 include a force transmission mechanism 1700 (see FIG. 5B) and an elongate shaft (see, e.g., shaft 2410 in FIGS. 6A-6C) that has one or more surgical end effectors (not shown in FIGS. 6A-6C) at a distal end. One or more actuation elements (such as rods, cables, or the like) that are coupled at a distal end to a wrist mechanism and/or the end effector (e.g., see the end effectors 4460 and 5460 described herein), and at a proximal end to the force transmission mechanism 1700 are used to control movements of the distal surgical tools. Rods, which may be in the form of tubular members, may be combined with cables to provide a “push/pull” control of the distal end tool end effector with the cables providing flexible sections as required. In some embodiments, the force transmission mechanism 1700 may be at a proximal end of the instrument, but the disclosure is not limited thereto or thereby, and in other embodiments, the force transmission mechanism 1700 may be positioned at an intermediate portion of the instrument between proximal and distal ends of the instrument.

In order to provide a sterile operation area while using a functional teleoperated surgical system, in some embodiments, optionally, a barrier is placed between non-sterile components of the system and the sterile surgical field. Therefore, a sterile component, such as an instrument sterile adapter (ISA) 1340, is optionally placed between the surgical instrument 1400 and the teleoperated surgical instrument manipulator unit 1200. The placement of an instrument sterile adapter 1340 between the surgical instrument 1400 and the surgical instrument manipulator unit 1200 includes the benefit of ensuring a sterile coupling point for the surgical instrument 1400 and the surgical instrument manipulator unit 1200. This permits removal of the surgical instruments 1400 from the surgical instrument manipulator unit 1200 and exchange with other surgical instruments during the course of a surgical procedure.

As shown in FIGS. 5B and 5C, the sterile adapter 1340 is releasably coupled to the instrument carriage 1330, and the force transmission mechanism 1700 of an instrument 1400 is releasably coupled to the sterile adapter 1340. More specifically, the sterile adapter 1340 includes a pair of latches 1305 (only one is shown in FIG. 5C) having latch receivers 1315. The force transmission mechanism 1700 includes a housing 1760 and a drive member interface 1725 coupled to a distal portion of the housing 1760. The drive member interface 1725 includes coupling portions (not shown) that are matingly couplable to the latches 1305 and have a connector that is received within the latch receivers 1315 of the latches 1305. For example, the latch receivers 1315 can couple to the coupling portions of the drive member interface 1725 with a snap connection. The latches 1305 can be squeezed to release the latches from the coupling portions of the drive member interface 1725 to decouple the robotic instrument 1400 from the sterile adapter 1340. In some embodiments, the latches 1305 and latch receivers 1315 of the sterile adapter 1340 and the coupling portions of the force transmission mechanism 1700 can be the same as or similar to, for example, the coupling mechanisms shown and described in U.S. Pat. No. 10,420,622 (filed Aug. 24, 2016), entitled “Latch to Secure Teleoperated Surgical Instrument to Actuator,” which is incorporated herein by reference in its entirety.

As shown in the exploded view of FIG. 5C, the instrument carriage 1330 includes a carriage interface 1335 that includes robotic drive outputs 1320 that are configured to be operatively coupled with inputs of the instrument at the drive member interface 1725. In embodiments utilizing a sterile adapter, the robotic drive outputs 1320 may be matingly coupled to couplers 1325 of the instrument sterile adapter 1340. The instrument carriage 1330 also includes an indentation or cutout region 1310 (shown in FIG. 5A in which the shaft (not shown) of the instrument 1400 can extend when the instrument 1400 is coupled to the system. In some embodiments, the robotic drive outputs 1320 of the carriage 1330 may be directly coupled to inputs of the drive member interface 1725 of the instrument without an intermediary sterile adapter 1340.

FIGS. 6A-6C are schematic illustrations of a robotic instrument 2400 (also referred to as “medical device”), a teleoperated system 2000, and a manual drive adapter 2800, according to an embodiment. FIG. 6A illustrates the robotic instrument 2400 releasably coupled to the teleoperated system 2000. FIG. 6B illustrates the robotic instrument 2400 detached from the teleoperated system 2000, and FIG. 6C illustrates the robotic instrument 2400 releasably coupled to the manual drive adapter 2800. In some embodiments, the teleoperated system 2000 can include the same or similar components and features as the system 1000 described above. For example, the teleoperated system 2000 can be used to perform surgical procedures, and can include a manipulator unit, a control unit, a series of kinematic linkages and joints, a series of cannulas, or the like. The medical device 2400 (and any of the instruments described herein) can be used in any suitable surgical system, such as the MIRS system 1000 shown and described above.

The robotic instrument 2400 includes a force transmission mechanism 2700 having a housing 2760, a drive member interface 2725 coupled to the housing 2760, and one or more input drive members 2710. The robotic instrument 2400 can be releasably coupled to a portion of the teleoperated system 2000, as described above for system 1000. The robotic instrument 2400 further includes a shaft 2410 coupled to the force transmission mechanism 2700, as shown in FIGS. 6A-6C. The robotic instrument 2400 can include one or more tools (e.g., end effectors, not shown but which can be similar to the end effectors 4460 and 5460 described herein) coupled to a distal end of the shaft 2410 that can be controlled with the teleoperated system 2000.

The teleoperated system 2000 includes one or more robotic drive outputs 2320 that can each be operatively coupled to a respective input drive member 2710 of the robotic instrument 2400 when the robotic instrument 2400 is coupled thereto. The drive member interface 2725 can include a coupling portion or portions (not shown) that can be releasably coupled to a portion of the teleoperated system 2000. For example, the teleoperated system 2000 can optionally include an instrument carriage (not shown, but which can be similar to the instrument carriage 1330 described above) within, or to which, the robotic drive output(s) 2320 can reside. In some embodiments, the instrument carriage can optionally be coupled to a sterile adapter (not shown) similar to or the same as the sterile adapter 1340 described above.

As previously described, during a surgical procedure using the teleoperated system 2000 and the robotic instrument 2400, it may be desirable to use a manually actuated medical device to perform some portion of the procedure. The manual drive adapter 2800 can be used for such a procedure. The robotic instrument 2400 can be detached from the teleoperated system 2000, as shown in FIG. 6B, and then releasably coupled to the manual drive adapter 2800 as shown in FIG. 6C. Thus, the same instrument (i.e., the robotic instrument 2400) can be used with both the teleoperated system 2000 and manually with the manual drive adapter 2800. As described in further detail below, the manual drive adapter 2800 is configured to couple with the same instrument outputs of the robotic instrument 2400 when the instrument 2400 is decoupled from the teleoperated system 2000.

The manual drive adapter 2800 includes an adapter housing 2810, an output drive interface 2815, and a manual actuator 2820 coupled to one or more output drive members 2822. The robotic instrument 2400 can be releasably coupled to the manual drive adapter 2800 in a similar manner as how the robotic instrument 2400 is coupled to the teleoperated system 2000. For example, in some embodiments, the input drive interface 2815 can include coupling portions (e.g., latches 1305 with latch receivers 1315) as described above for sterile adapter 1340. The coupling portions of the input drive interface 2815 of the manual drive adapter 2800 can be coupled to mating coupling portions of the drive member interface 2725 of the robotic instrument 2400. When the robotic instrument 2400 is coupled to the manual drive adapter 2800, the input drive member(s) 2710 engage with the output drive members 2822 of the manual drive adapter 2800 such that the robotic instrument can be actuated using the manual actuator 2820. Thus, the robotic instrument 2400 can be releasably coupled to the teleoperated system 2000 and controlled by the teleoperated instrument 2000, and releasably coupled to the manual drive adapter 2800 and be controlled by the manual actuator 2820 when unattached from the teleoperated system 2000.

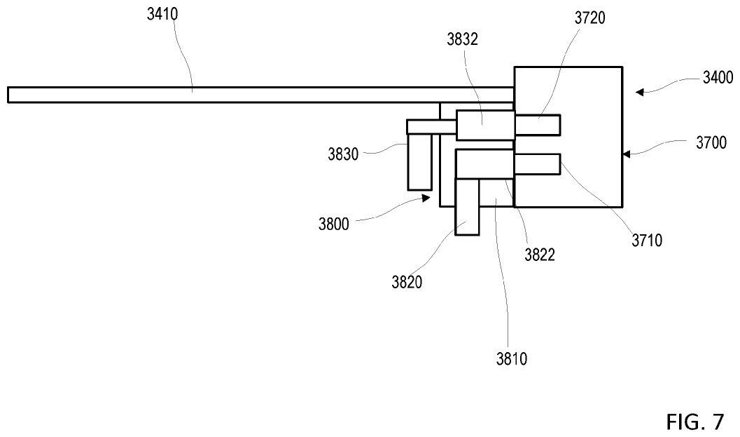

FIG. 7 illustrates a robotic instrument 3400 and a manual drive adapter 3800, according to another embodiment. As with other embodiments described herein, the robotic instrument 3400 can be releasably coupled to a teleoperated system (e.g., systems 1000, 2000), and also releasably coupled to the manual drive adapter 3800 when detached from the teleoperated system (not shown in FIG. 7).

The robotic instrument 3400 includes a force transmission mechanism 3700, a first input drive member 3710, and a second input drive member 3720 coupled to, or disposed within, for example, a housing of the force transmission mechanism 3700. In some embodiments, the robotic instrument 3400 can optionally include a shaft 3410 coupled to the force transmission mechanism 3700. The robotic instrument 3400 can include one or more wrist mechanisms and/or end effectors (e.g., similar to the end effectors 4460 and 5460 described herein) coupled to a distal end of the shaft 3410 that can be controlled with the teleoperated system (e.g., the teleoperated system 1000 or 2000 described above) and/or with the manual drive adapter 3800.

The manual drive adapter 3800 includes an adapter housing 3810, a first manual actuator 3820 coupled to a first output drive member 3822, and a second manual actuator 3830 coupled to a second output drive member 3832. The robotic instrument 3400 can be releasably coupled to the manual drive adapter 3800 in a similar manner as how the robotic instrument 3400 is coupled to the teleoperated system, as described for other embodiments. For example, in some embodiments, the manual drive adapter 3800 can include an output drive interface (not shown) that can include coupling portions (e.g., latches 1305 with latch receivers 1315) as described above for sterile adapter 1340. The coupling portions of the manual drive adapter 3800 can be coupled to mating coupling portions of a drive member interface (not shown) of the robotic instrument 3400. When the robotic instrument 3400 is coupled to the manual drive adapter 3800, the first input drive member 3710 engages with the first output drive member 3822 of the manual drive adapter 3800 and the second input drive member 3720 engages with the second output drive member 3832 of the manual drive adapter 3800 such that the robotic instrument 3400 can be actuated using the first manual actuator 3820 and the second manual actuator 3830. Thus, the robotic instrument 3400 can be releasably coupled to a teleoperated system and controlled by the teleoperated instrument, and releasably coupled to the manual drive adapter 3800 and controlled by the manual actuator 3820 when the robotic instrument 3400 is detached from the teleoperated system.

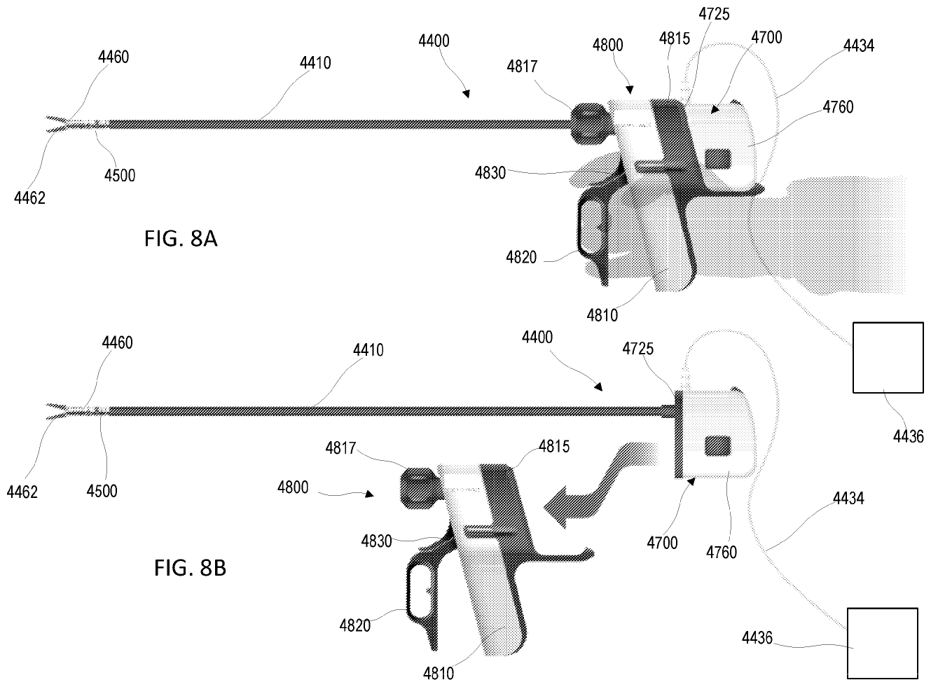

FIGS. 8A and 8B illustrate another embodiment of a manual drive adapter 4800 and a robotic instrument 4400 coupled to the manual drive adapter 4800 in FIG. 8A and decoupled from the manual drive adapter 4800 in FIG. 8B. The robotic instrument 4400 can be releasably coupled to and operated by a teleoperated system as described herein for other embodiments. The robotic instrument 4400 can also be releasably coupled to the manual drive adapter 4800 and operated and controlled by manual actuators of the manual drive adapter 4800. For example, as described herein, during a procedure using the robotic instrument 4400 coupled to and operated by a teleoperated system, the surgeon may wish to disconnect from the teleoperated system and perform some portions of a procedure using a manually actuated device. The robotic instrument 4400 can be disconnected from the teleoperated system and releasably coupled to the manual drive adapter 4800. In some embodiments, the manual drive adapter 4800 can be provided in a sterile kit and provided for a single use. For example, in some embodiments, the manual drive adapter 4800 can be stored within a sterile package prior to use. The package can be, for example, a sealed biocompatible plastic package that maintains the manual drive adapter 4800 in a sterile environment prior to use. In some embodiments the package can be constructed from a material that prevents microbial transfer into the packaging. In use, the manual drive adapter 4800 can first be removed from the sterile package and coupled to the robotic instrument, as described above. In other embodiments, the manual drive adapter 4800 (and any of the manual drive adapters described herein) can include a sterile drape or other suitable barrier to maintain the robotic instrument 4400 and/or the coupling location thereof in a sterile environment apart from the manual actuators. In other embodiments, the manual drive adapter 4800 (and any of the manual drive adapters described herein) can be coupled to a separate sterile adapter placed between the robotic instrument 4400 and the manual drive adapter 4800. In still other embodiments, the manual drive adapter 4800 can be provided for multiple use. For example, the manual drive adapter 4800 can be configured to be cleaned and sterilized to be reused for multiple procedures. In some embodiments, sterile draping may not be required.

The robotic instrument 4400 includes a force transmission mechanism 4700, a drive member interface 4725 coupled to the force transmission mechanism 4700, and a shaft 4410. The force transmission mechanism 4700 includes a housing 4760, and a first input drive member (not shown) and a second input drive member (not shown) coupled within the housing 4760. In some embodiments, the force transmission mechanism 4700 may have additional input drive members (e.g., three, four, five, or a different amount of input drive members). The shaft 4410 has a proximal end coupled to the force transmission mechanism 4700 and a distal end coupled to a wrist mechanism 4500 and end effector 4460. The end effector 4460 includes a first tool 4462 that includes a gripping tool and a second tool (not shown) that includes a cutting tool; however, other tools may be included additionally or alternatively to first tool and second tool. The cutting tool can be a mechanically actuated blade, or an electric cautery tool. The first tool 4462 includes a pair of jaws that open and close to perform a clamping operation, and the second tool is disposed between the opposing jaws and translates in proximal and distal directions along a longitudinal axis of the jaws (also referred to as the z-axis). The wrist mechanism 4500 provides for articulating motion and the shaft 4410 can be rotated. The jaws of the gripping tool can operate to grip tissue and additionally can be configured to deliver electrosurgical energy to fuse tissue together, for example, to fuse tissue of a dissected vessel in order to seal the ends of the dissected vessel. Each jaw may optionally include an electrode that receives energy from an associated electrical conductor. An embodiment of a medical device having a gripping tool and cutting tool is described below with reference to robotic instrument 5400. Other example embodiments of such medical devices are described in U.S. Pat. No. 9,055,961 (filed Feb. 17, 2012), entitled “Fusing and Cutting Surgical Instrument and Related Methods” and U.S. Pat. No. 9,043,027 (filed May 30, 2012), entitled “Positive Control of Robotic Surgical Instrument End Effector,” and U.S. Patent Publication No. 20210177495, entitled “Electrosurgical Instruments for Sealing and Dissecting” (filed Oct. 30, 2020), the disclosure of each of which is incorporated herein by reference in its entirety.

The manual drive adapter 4800 includes an adapter housing 4810, an output drive interface 4815 coupled to the adapter housing 4810, a first manual actuator 4820 coupled to a first output drive member (not shown) within the adapter housing 4810, and a second manual actuator 4830 coupled to a second output drive member (not shown) within the adapter housing 4810. The manual drive adapter 4800 also includes a rotating knob 4817 that can be coupled to the shaft 4410 and used to manually rotate the shaft 4410 about a longitudinal axis of the shaft 4410 relative to the adapter housing 4810 and lock the shaft 4410 in position. In some embodiments, the knob 4817 can drive the roll input of the shaft 4410 by means of a series of gears (or cables, belts, etc.). In an alternative embodiment, the rotating knob 4817 can be coupled to the instrument input that controls the shaft roll, rather than being directly coupled to the shaft 4410. The robotic instrument 4400 can be releasably coupled to a teleoperated system (not shown, but which can be similar to the teleoperated systems 1000 and 2000), and when decoupled from the teleoperated system, releasably coupled to the manual drive adapter 4800 in a similar manner as described herein for other embodiments. For example, in some embodiments, the output drive interface 4815 of the manual drive adapter 4800 can include coupling portions (e.g., latches 1305), as described above for sterile adapter 1340. The coupling portions can be coupled to mating coupling portions of the drive member interface 4725 of the robotic instrument 4400.

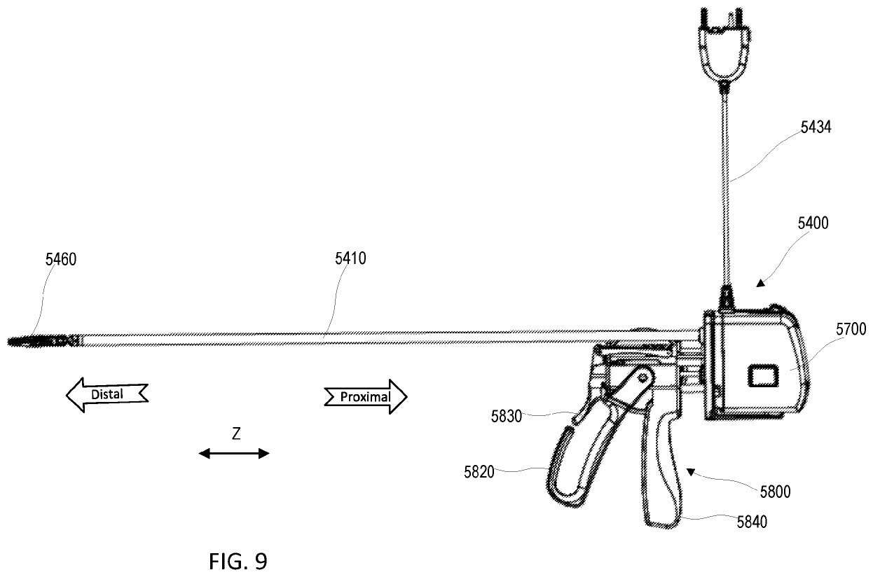

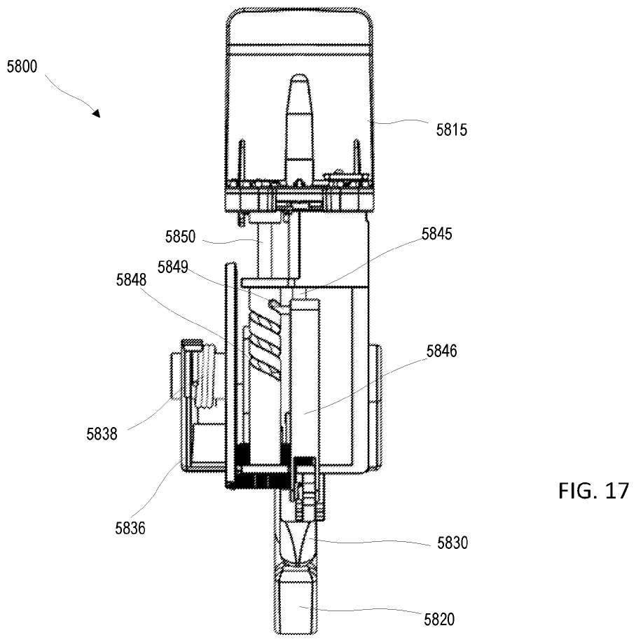

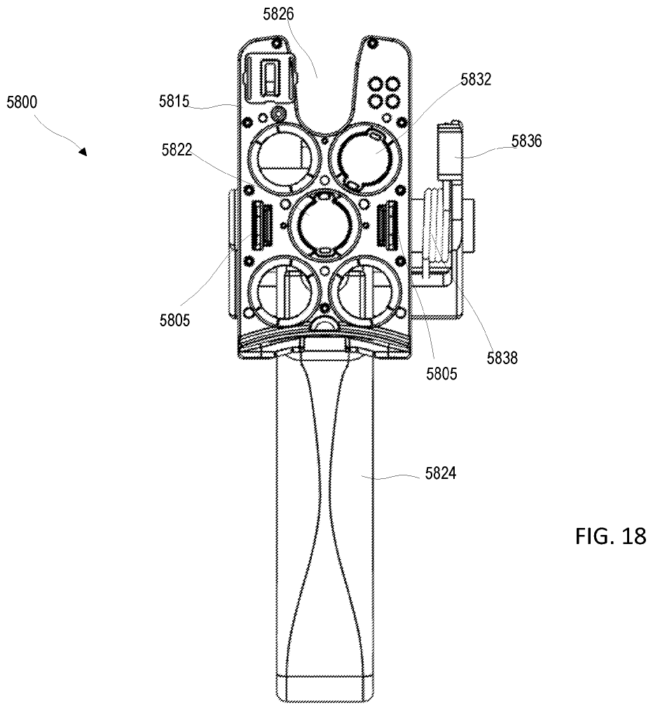

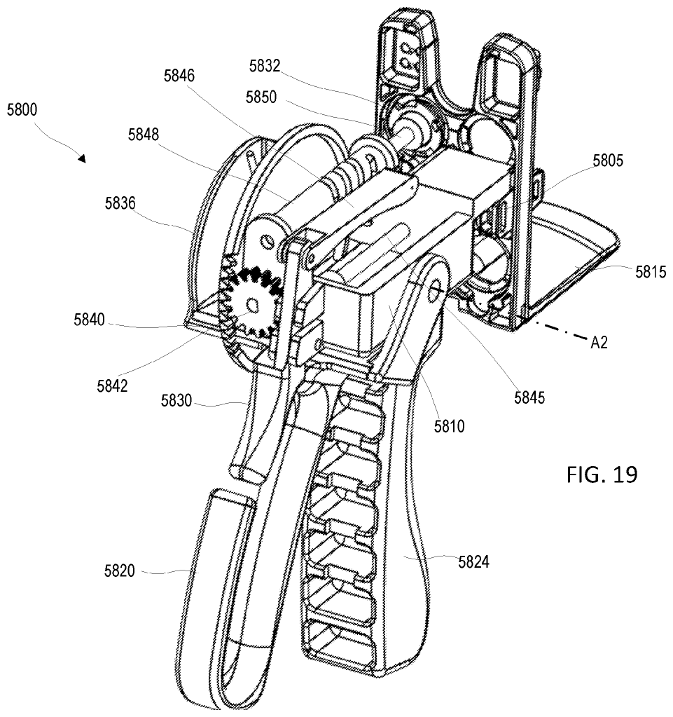

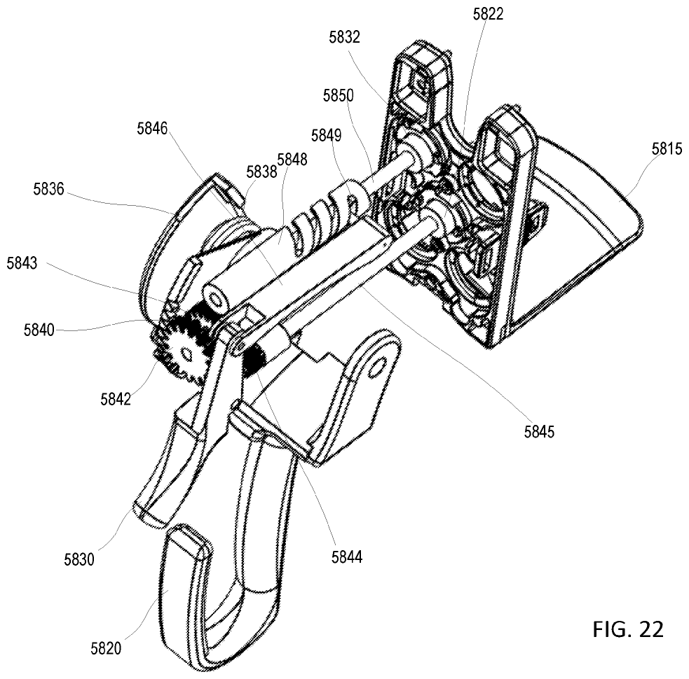

When the robotic instrument 4400 is coupled to the manual drive adapter 4800, the first input drive member of the robotic instrument 4400 engages with the first output drive member of the manual drive adapter 4800 and the second input drive member of the robotic instrument 4400 engages with the second output drive member of the manual drive adapter 4800 such that the robotic instrument 4400 can be actuated using the first manual actuator 4820 and the second manual actuator 4830. More specifically, the first manual actuator 4820 can be used to actuate the first tool 4462 (e.g., for closing the jaws of the instrument 4400 and applying gripping force) and the second manual actuator can be used to actuate the second tool (e.g., for cutting once the jaws have been closed) (not shown). Thus, movement of the first manual actuator 4820 by the user causes the first output drive member to move the first input drive member of the robotic instrument 4400 to actuate the first tool 4462 of the end effector 4460. Likewise, movement of the second manual actuator 4820 by the user causes the second output drive member to move the second input drive member of the robotic instrument 4400 to actuate the second tool of the end effector 4460. When the output drive interface 4815 of the manual drive adapter 4800 is coupled to the drive member interface 4725 of the robotic instrument 4400, the input drive members of the robotic instrument 4400 are prevented from being engaged with robotic drive outputs of a teleoperated surgical system. Further details regarding components and actuation of a manual drive adapter 5800 are provided below with reference to FIGS. 9-22. The manual drive adapter 4800 can include the same or similar components and functions as the manual drive adapter 5800.

In some embodiments, the wrist mechanism 4500 of the robotic instrument 4400 includes a link (not identified in FIGS. 8A and 8B) that is coupled to the distal end of the shaft 4410 and coupled to the first tool 4462 and/or the second tool (not shown). The link, which can be a clevis or other suitable kinematic link of the wrist mechanism 4500, is configured to rotate about a rotation axis to move a position of the first tool 4462 and/or second tool about the rotation axis (e.g., in a pitch and/or yaw motion). In some embodiments, the manual drive adapter 4800 includes a third manual actuator (not shown in FIGS. 8A and 8B). The third manual actuator can be configured to be manually actuated by the user to lock the link in a fixed position relative to the shaft 4410 and prevent at least one of the first tool 4462 or the second tool from moving about the rotation axis. In some embodiments, the manual drive adapter 4800 has a lock mechanism that is incorporated into any of the manual actuators (e.g., the first manual actuator 4820 or the second manual actuator 4830) or other portions of the manual drive adapter 4800 described herein. The lock mechanism can include a material within the manual actuator, the output drive interface 4815, and/or the coupling (that mate to the coupling portions of the drive member interface 4725) that has a high coefficient of friction (e.g., a rubberized material) such that it holds the wrist in place by way of friction. In some embodiments, the friction can be high enough such that the wrist can also be prevented from being moved even by an external or backdrive force. In some embodiments, the lock component can be configured with a controlled amount of friction that allows the wrist to be held in place, and also allows for the wrist to be adjusted by the user (e.g., via backdrive force or movement of a manual actuator). In yet other embodiments, the manual drive adapter 4800 has a third manual actuator that can be manually actuated by the user to actuate the wrist.

In some embodiments, the manual drive adapter 4800 can optionally include a lockout mechanism (not shown in FIGS. 8A and 8B) that prevents the second manual actuator 4830 from being actuated unless the first manual actuator 4820 is actuated. For example, the lockout mechanism can prevent actuation of a cutting mechanism until the jaws of the end effector are closed. In some embodiments, the manual drive adapter 4800 can include a torque limiter (not shown in FIGS. 8A and 8B) coupled to the first manual actuator 4820 and configured to limit a torque applied to the first output drive member when the first manual actuator is moved by the user. In some embodiments, the torque limiter includes a torsion spring. Such an embodiment is described below with reference to manual drive adapter 5800. In some embodiments, the torque limiter can include a torsion spring as described in U.S. Pat. No. 9,913,694, entitled “Grip Force Control in a Robotic Surgical Instrument,” issued Mar. 13, 2018, the entire disclosure of which is incorporated herein by reference.

The robotic instrument 4400 can also include a power cord 4434 that can be connected to a power source 4436 such that when the manual drive adapter 4800 is coupled to the robotic instrument 4400, the first tool and/or the second tool can be electrically coupled to the power source 4436. The power source 4436 can be included with the teleoperated system or provided separately. Thus, the robotic instrument 4400 can be releasably coupled to a teleoperated system and controlled by the teleoperated instrument, and releasably coupled to the manual drive adapter 4800 and controlled by the manual actuator 4820 and 4830. Likewise, the end effector (and tools) can be controlled by a teleoperated system when the robotic instrument 4400 is coupled thereto and can also be controlled by the manual drive adapter 4800 when the robotic instrument 4400 is coupled thereto.

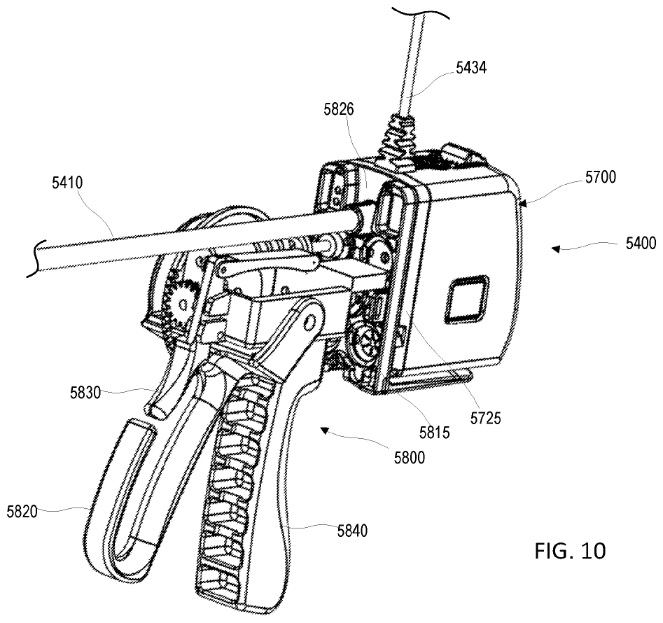

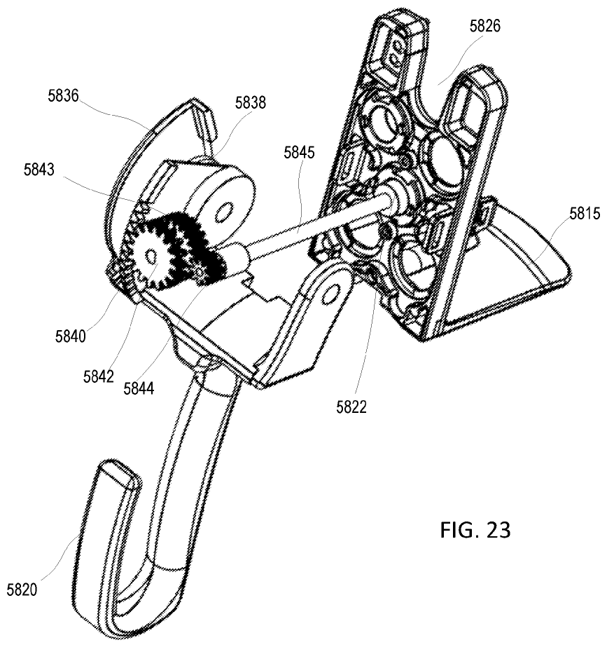

FIGS. 9-24B illustrate a robotic instrument 5400 and a manual drive adapter 5800, according to another embodiment. FIGS. 9 and 10 show the robotic instrument 5400 coupled to the manual drive adapter 5800. The robotic instrument 5400 can be releasably coupled to and operated by a teleoperated system (not shown), as described herein for other embodiments. The robotic instrument 5400 can also be releasably coupled to the manual drive adapter 5800 and operated and controlled by manual actuators of the manual drive adapter 5800 described in more detail below.

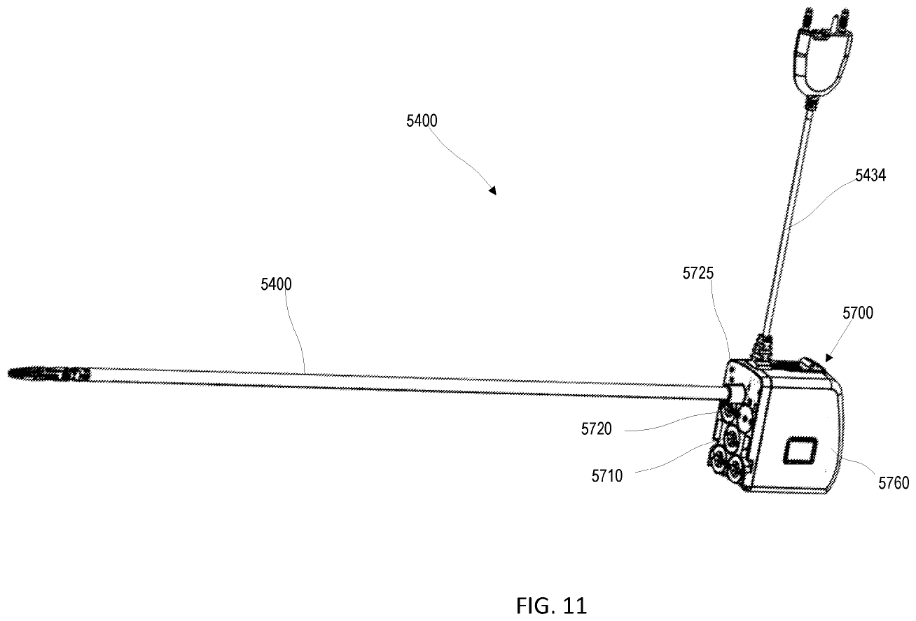

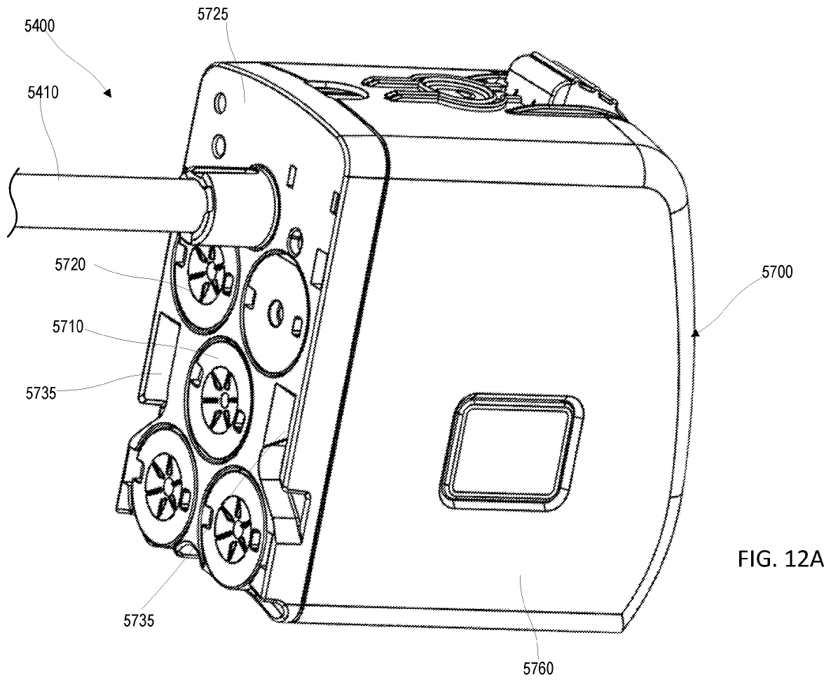

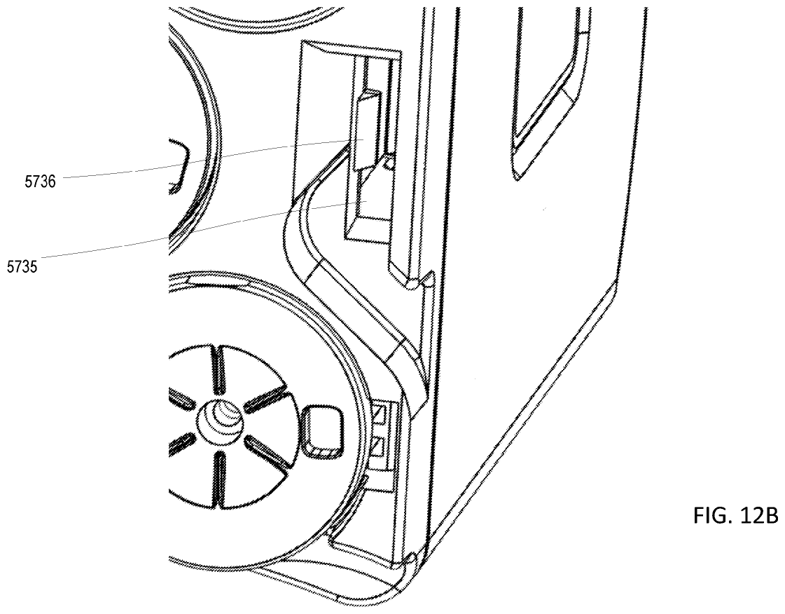

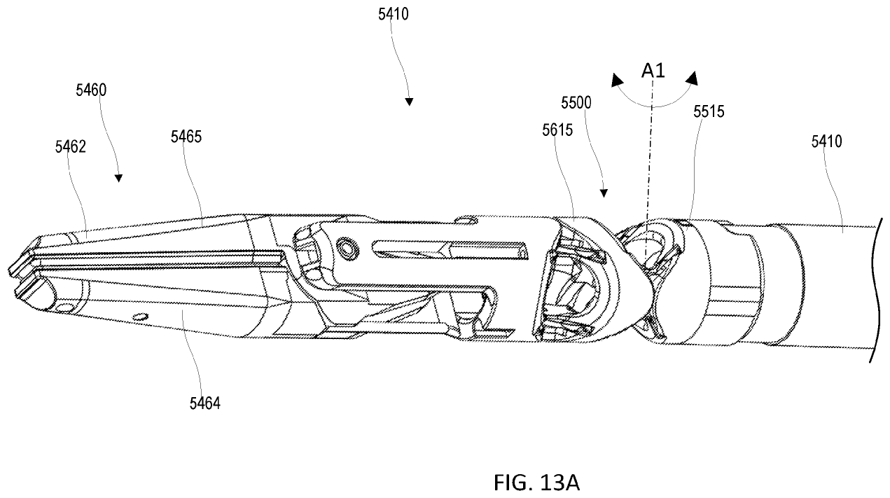

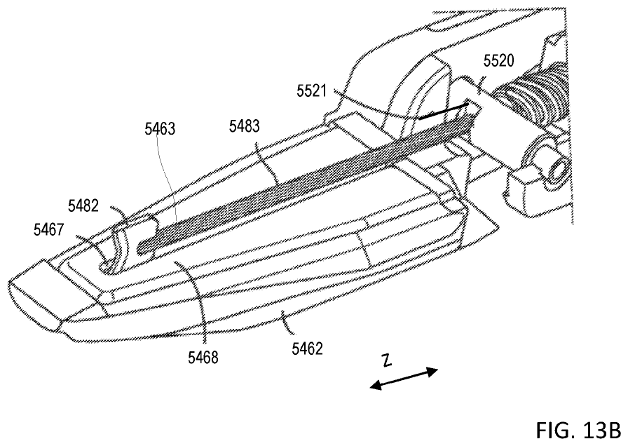

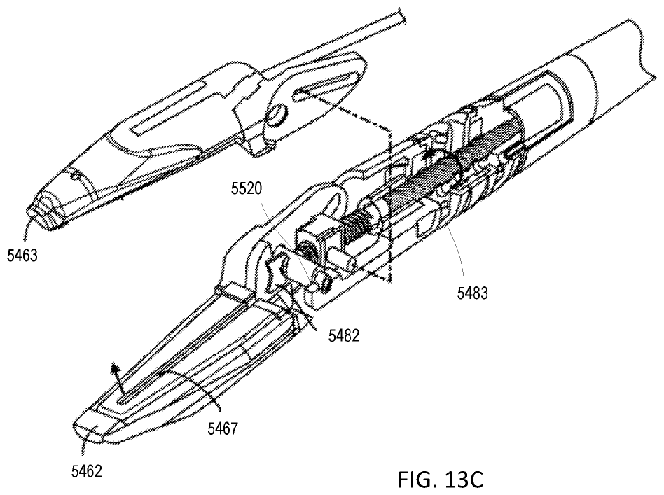

The robotic instrument 5400 includes a force transmission mechanism 5700, a drive member interface 5725 coupled to the force transmission mechanism 5700, a wrist mechanism 5500 (see FIG. 13A), and a shaft 5410. The force transmission mechanism 5700 includes a housing 5760, and multiple input drive members coupled to, or disposed at least partially within, the housing 5760. A plurality of input drive members, including a first input drive member 5710 and a second input drive member 5720, are depicted in FIGS. 11 and 12A and discussed in more detail below. The shaft 5410 includes a proximal end coupled to the force transmission mechanism 5700 and a distal end coupled to the wrist mechanism 5500.

As shown in FIGS. 13A-13C, the end effector 5460 includes a first tool 5462 that includes a gripping tool and a second tool 5463 (see FIG. 13B) that includes a cutting element 5482. The cutting tool can be an electric cautery cutting tool. The first tool 5462 includes a pair jaws 5464 and 5465 that open and close to perform a clamping operation. The jaws 5464 and 5465 of the gripping tool can operate to grip tissue and additionally and/or optionally can be configured to deliver electrosurgical energy to fuse tissue together, for example, to fuse tissue of a dissected vessel in order to seal the ends of the dissected vessel. For example, each jaw 5464 and 5465 may optionally include an electrode (not shown) that receives energy from an associated electrical conductor The second tool 5463 is disposed between the opposing jaws 5464 and 5465 of the first tool 5462 and translates in proximal and distal directions along a longitudinal z-axis direction. The second tool 5463 includes a cutting element 5482 and a cutting element drive component 5483. When the second tool 5463 is actuated, the cutting element 5482 is moved between a proximal position in which the cutting element 5482 is disposed within a notch in 5521 in a proximal clevis pin 5520 (see FIG. 13B), and a distal position in which the cutting element 5482 is disposed at a distal end of a groove 5467 in the jaw 5462 and a corresponding groove (not shown in the jaw 5463, Other example embodiments of a medical device having a gripping tool and cutting tool are described in U.S. Pat. Nos. 9,055,961 and 9,043,027, and U.S. Publication No. 20210177495, the disclosures of which are incorporated by reference herein above.

The wrist mechanism 5500 of the robotic instrument 5400 includes a proximal link 5515 rotatably coupled to a distal link 5615 (see FIG. 13A). The distal link 5615 is coupled to the distal end of the shaft 5410 via the proximal link 5515 and is coupled to the first tool 5462 and the second tool 5463. The distal link 5615 is configured to rotate about a rotation axis A1 relative to the proximal link 5515 to move a position of the first tool 5462 and second tool 5463. One or more actuation elements (not shown), such as cables, are coupled to the input drive members 5710, 5720, extend through the shaft 5410 and the wrist mechanism 5500, and are coupled to the end effector 5460. The cables can be used to control movements of the end effector 5460. For example, when the input drive members 5710 and 5720 are actuated (e.g., moved), the cables in turn cause movement at the end effector 5460. It should be understood that the input drive members 5710 and 5720 are by way of example and that in some embodiments, additional input drive members of the interface 5725 can be coupled to the end effector 5460. Further, additional actuation elements can be coupled to additional input drive members of the force transmission mechanism 5700 and coupled to the wrist mechanism to control movements of the wrist mechanism.

The robotic instrument 5400 can also include a power cord 5434 that can be connected to a power source (not shown) such that when the manual drive adapter 5800 is coupled to the robotic instrument 5400, the first tool 5462 and/or the second tool 5463 can be electrically coupled to the power source. The power source can be included with the teleoperated system or provided separately.

The robotic instrument 5400 can be releasably coupled to a teleoperated system and to the manual drive adapter 5800 in a similar manner as described herein for other embodiments. As described for other embodiments, the drive member interface 5725 of the robotic instrument 5400 can be coupled to the output drive interface 5815 of the manual drive adapter 5800 as described in more detail below. When the drive member interface 5725 of the robotic instrument 5400 is coupled to the input drive interface 5815 of the manual drive adapter 5800, the input drive members 5710 and 5720 of the robotic instrument 5400 are prevented from being engaged with robotic drive outputs of a teleoperated surgical system (not shown). The input drive members 5710, 5720 can be actuated using robotic actuators of the teleoperated system when coupled thereto and can be actuated by manual actuators of the manual drive adapter 5800 when coupled thereto, as described in more detail below.

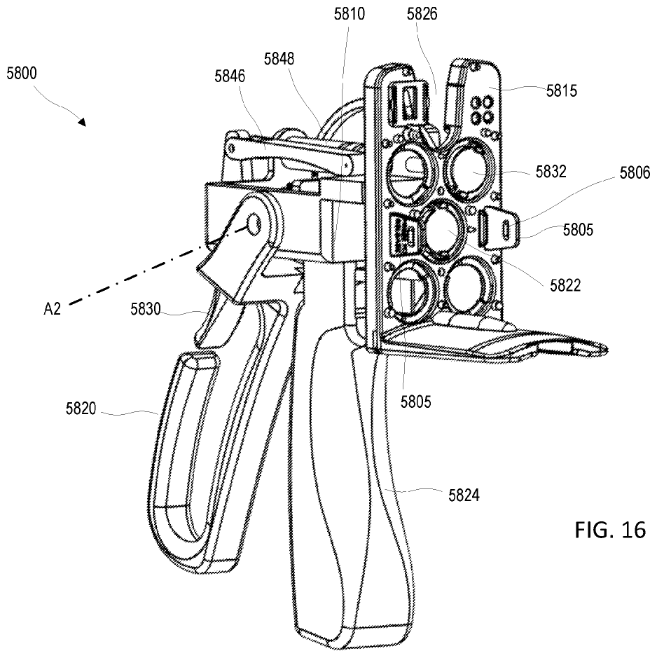

The manual drive adapter 5800 includes an adapter housing 5810, an output drive interface 5815 coupled to the adapter housing, a grip handle 5824, a first manual actuator 5820 coupled to a first output drive member 5822, and a second manual actuator 5830 coupled to a second output drive member 5832 (see, e.g., FIGS. 16, 18 and 21). Although the adapter housing 5810 is shown as being coupled to the grip handle 5824 by one or more rods or structures, in other embodiments, the adapter housing 5810 and the grip handle 5824 can be monolithically constructed (e.g., similar to the structure of the manual drive adapter 4800 described above). The manual drive adapter 5800 can also optionally include a rotating knob (not shown) that can be coupled to the shaft 5410 and used to manually rotate the shaft 5410 about a longitudinal axis of the shaft 5410 relative to the adapter housing and lock the shaft 5410 in position. As described above for the previous embodiment, the rotating knob can alternatively be coupled to the instrument input that controls the shaft roll. In some embodiments, the knob can drive the roll input of the shaft 4410 by means of a series of gears (or cables, belts, etc.). The output drive interface 5815 of the manual drive adapter 5800 includes latches 5805 having latch receivers 5806. The latches 5805 and latch receivers 5806 can be the same as or similar to the latches 1305 and latch receivers 1315 described above for sterile adapter 1335. The latches 5805 can be used to releasably couple the robotic instrument 5400 to the manual drive adapter 5800 in a similar manner as described above. More specifically, the drive member interface 5725 of the force transmission mechanism 5700 includes coupling portions 5735 (see FIGS. 12A and 12B) that receive the latches 5805, and an inner connector 5736 that is received within the latch receivers 5806 (see FIG. 12B). For example, the latch receivers 5806 can couple to the inner connectors 5736 of the drive member interface 5725 with a snap connection. The latches 5805 can be squeezed to release the inner connectors 5736 from the latch receivers 5806 to decouple the robotic instrument 5400 from the manual drive adapter 5800. In some embodiments, the latches 5805 and latch receivers 5806 of the manual drive adapter 5800 and the coupling portions 5735 and inner connectors 5736 of the force transmission mechanism 5700 can be the same as or similar to, for example, the coupling mechanisms shown and described in U.S. Pat. No. 10,420,622, which is incorporated herein above by reference in its entirety. As shown in FIG. 16, the output drive interface 5815 also includes an indentation or cutout region 5826 in which the shaft 5410 of the robotic instrument 5400 can extend when the robotic instrument 5400 is coupled to the manual drive adapter 5800.

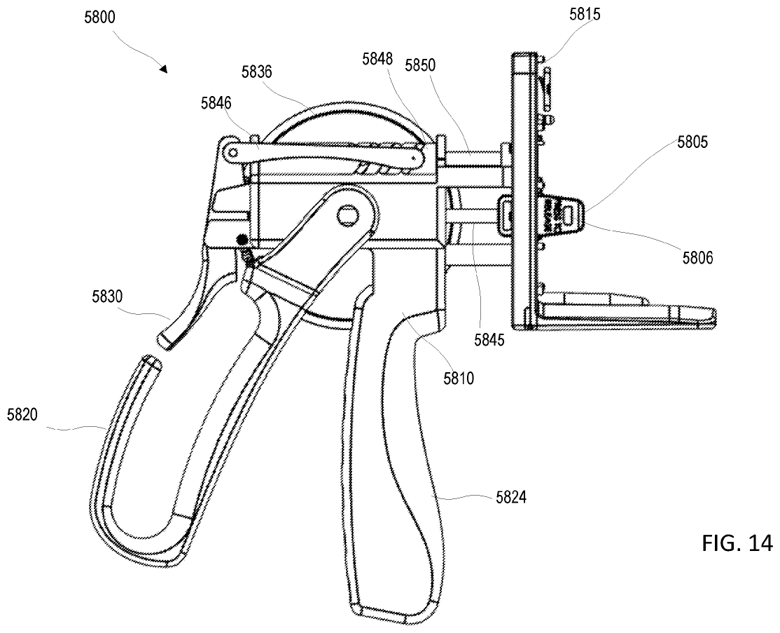

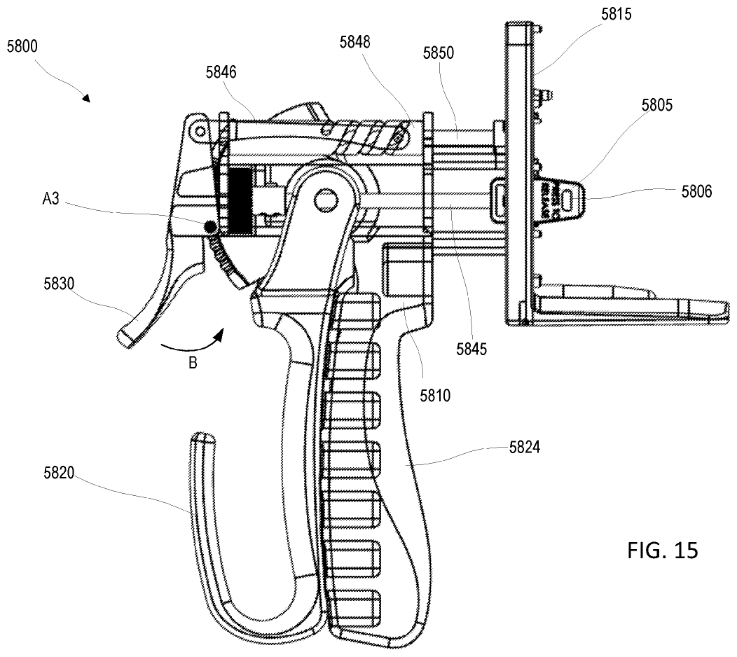

When the robotic instrument 5400 is coupled to the manual drive adapter 5800, the first input drive member 5710 of the robotic instrument 5400 engages with the first output drive member 5822 of the manual drive adapter 5800 and the second input drive member 5720 of the robotic instrument 5400 engages with the second output drive member 5832 of the manual drive adapter 5800 such that the robotic instrument 5400 can be actuated using the first manual actuator 5820 and the second manual actuator 5830. More specifically, the first manual actuator 5820 can be used to actuate the first tool 5462 of the robotic instrument 5400 and the second manual actuator 5830 can be used to actuate the second tool 5463 of the robotic instrument 5400. Thus, movement of the first manual actuator 5820 by the user causes the first output drive member 5822 to move (i.e., rotate) the first input drive member 5710 of the robotic instrument 5400 to actuate the first tool 5464 of the end effector 5460. Likewise, movement of the second manual actuator 5820 by the user causes the second output drive member 5832 to move (i.e., rotate) the second input drive member 5720 of the robotic instrument 5400 to actuate the second tool 5465 of the end effector 5460. FIG. 14 illustrates the first manual actuator 5820 in a ready position, and FIG. 15 illustrates the first manual actuator 5820 in an actuated position (moved by a user).

In some embodiments, the manual drive adapter 4800 has a lock mechanism that is incorporated into any of the manual actuators (e.g., the first manual actuator 4820 or the second manual actuator 4830) or other portions of the manual drive adapter 4800 described herein. The lock mechanism can include a material within the manual actuator, the output drive interface 4815, and/or the coupling (that mate to the coupling portions of the drive member interface 4725) that has a high coefficient of friction (e.g., a rubberized material) such that it holds the wrist in place by way of friction. In some embodiments, the friction can be high enough such that the wrist can also be prevented from being moved even by an external or backdrive force. In some embodiments, the lock component can be configured with a controlled amount of friction that allows the wrist to be held in place, and also allows for the wrist to be adjusted by the user (e.g., via backdrive force or movement of a manual actuator). In yet other embodiments, the manual drive adapter 4800 has a third manual actuator that can be manually actuated by the user to actuate the wrist.

In some embodiments, the manual drive adapter 5800 includes a third manual actuator (not shown) that can be configured to be manually actuated by the user to lock the link and/or the wrist mechanism 5500 in a fixed position relative to the shaft 5410 and prevent at least one of the first tool 5462 or the second tool 5463 from moving about the rotation axis. As described above for the previous embodiment, the manual drive adapter 5800 can include a lock mechanism that is incorporated into any of the manual actuators (e.g., the first manual actuator 5820 or the second manual actuator 5830) or other portions of the manual drive adapter 5800 described herein. The lock mechanism can include a material within the manual actuator, the output drive interface 5815, and/or the coupling (that mate to the coupling portions of the drive member interface 5725) that has a high coefficient of friction (e.g., a rubberized material) such that it holds the wrist in place by way of friction. In some embodiments, the friction can be high enough such that the wrist can also be prevented from being moved even by an external or backdrive force. In some embodiments, the lock component can be configured with a controlled amount of friction that allows the wrist to be held in place, and also allows for the wrist to be adjusted by the user (e.g., via backdrive force or movement of a manual actuator). In yet other embodiments, the manual drive adapter 5800 has a third manual actuator that can be manually actuated by the user to actuate the wrist.

Further, the manual drive adapter 5800 can optionally include a lockout mechanism (not shown) that prevents the second manual actuator 5830 from being actuated unless the first manual actuator 5820 is actuated. In other words, to actuate the second manual actuator 5830, the first a manual actuator 5820 must be in the actuated/moved position as shown in FIG. 15.

As described above, the first manual actuator 5820 is coupled to the first output drive member 5822 and the second manual actuator 5830 is coupled to the second output drive member 5832. The first output drive member 5822 can actuate movement of the first tool 5462 and the second output drive member 5832 can actuate movement of the second tool 5463. As described above and as shown, for example, in FIGS. 13A-13C, the first tool 5462 includes a pair of jaws 5464 and 5465 and can be used as a gripping tool, and the second tool 5463 includes a cutting element 5482.

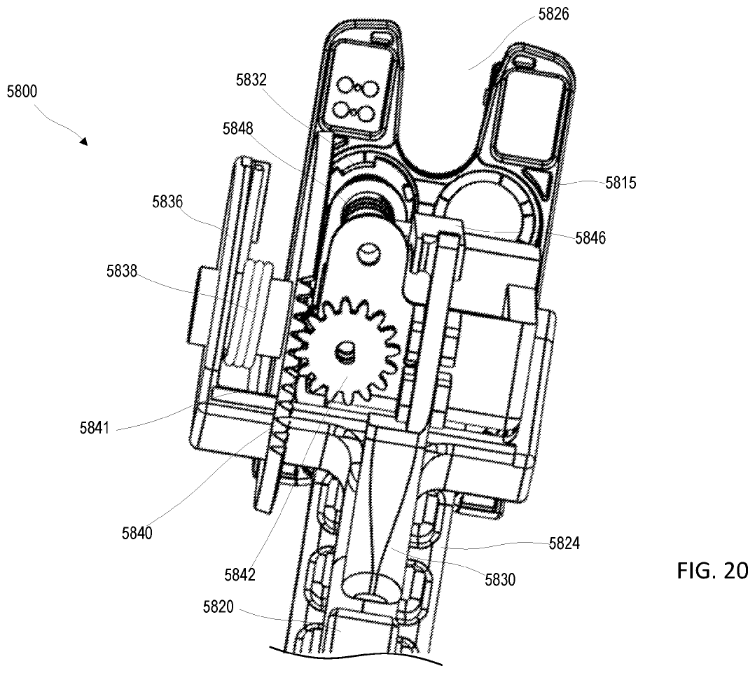

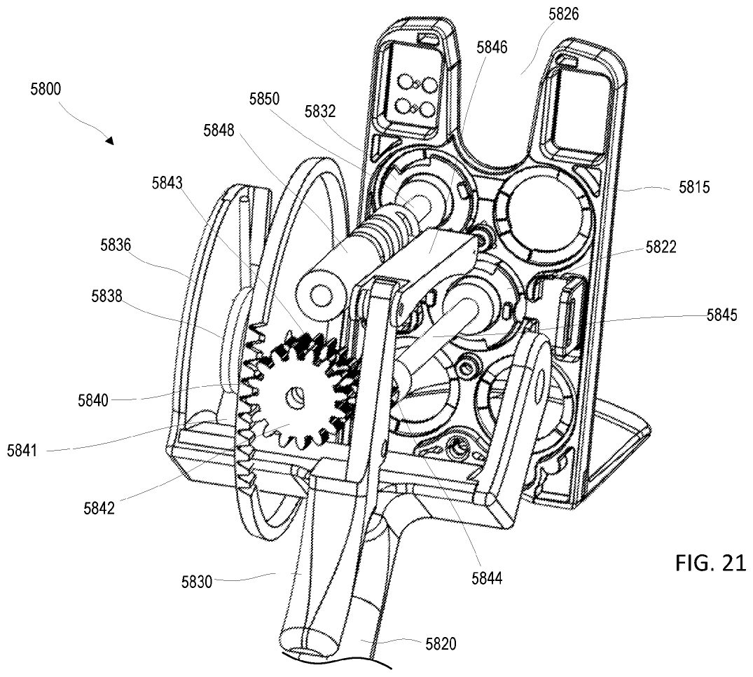

To actuate and move the first tool 5462 (e.g., gripping tool) using the manual drive adapter 5800, the user moves (e.g., squeezes) the first manual actuator 5820 towards the grip handle 5824 as shown in FIG. 15, such that the first manual actuator 5820 rotates about an axis A2 (see FIG. 16). The first manual actuator 5820 is coupled to or is an integral part of a torsion spring driver component 5836, which is coupled to a torsion spring 5838. As the first manual actuator 5820 is rotated, the torsion spring driver component 5836 activates the torsion spring 5838 to maintain a constant and limited grip pressure at the first tool 5462. Thus, the torsion spring 5838 functions as a torque limiter. In alternative embodiments, other types of torque limiters can be used. The torsion spring 5838 contacts a crown gear 5840 at a contact point 5841 (see FIG. 20), which drives the crown gear 5840. The crown gear 5840 then drives three transmission gears 5842, 5843 and 5844 (see e.g., FIGS. 21-23), which are coupled to a first output drive shaft 5845 (e.g., a gripper drive shaft). The gripper drive shaft 5845 is coupled to the output drive member 5822, which in turn drives the first output drive member 5710 of the robotic instrument 5400. In this manner, rotation of the first manual actuator 5820 about axis A2 causes rotation of the gripper drive shaft 5845 and the output drive member 5822, which, in turn, rotates the first output drive member 5710. As described above, the first output drive member 5710 is coupled to actuation elements (e.g., cables) that are coupled to the end effector 5460 and first tool 5462 and can control movements of the first tool 5462 (e.g., gripper tool).

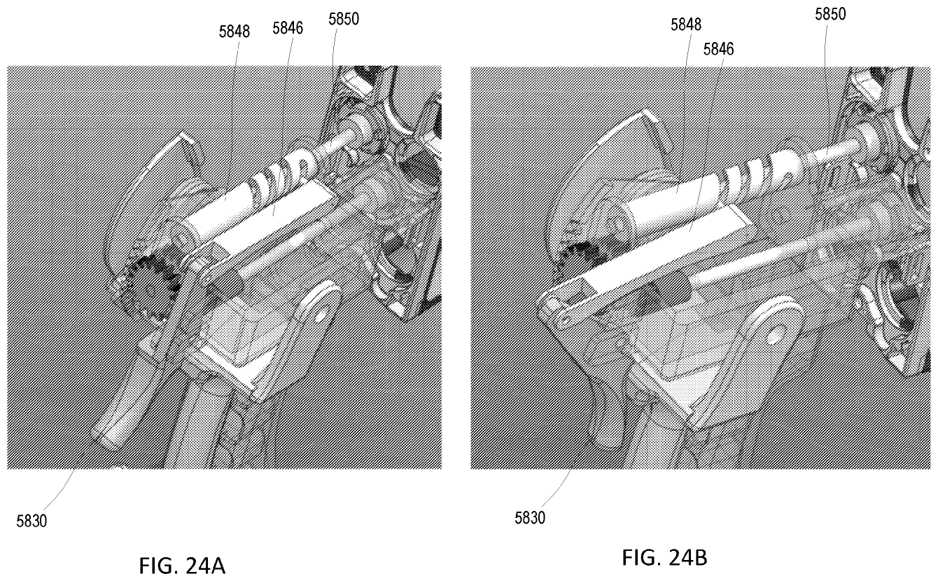

To actuate and move the second tool 5463 (e.g., cutting tool) using the manual drive adapter 5800, the user must first move the first manual actuator 5820 to an actuated position as shown in FIG. 15. Holding the first manual actuator 5820 in the actuated position, the user can move the second manual actuator 5830 in a direction toward the first manual actuator 5820. In other words, the user moves the second manual actuator 5830 such that it rotates in a direction of arrow B, shown in FIG. 15, about an axis A3 (see FIG. 15). FIG. 24A illustrates the second manual actuator 5830 in a ready position and FIG. 24B illustrates the second manual actuator 5830 in an actuated position.

When the second manual actuator 5830 is rotated about the axis A3, the second manual actuator 5830 drives a linkage 5846 coupled to the second manual actuator 5830 to move the linkage 5846 substantially linearly. The linkage 5846 includes pin 5849 that is disposed within a helical groove of a helix member 5848 coupled to a second output drive shaft 5850 (e.g., a cutter driver). Thus, when the linkage 5846 is moved (see, e.g., FIGS. 24A and 24B), corresponding movement of the pin 5849 coupled to the helical groove of the helix member 5848 causes the helix member 5848 and therefore the cutter driver 5850 to rotate. In this manner, rotation of the second manual actuator 5830 causes linear motion of the linkage 5846, which is converted into rotational motion of the cutter driver 5850 (see, e.g., FIGS. 24A and 24B). The cutter driver 5850 is coupled to the second output drive member 5832, which in turn drives the second output drive member 5720 of the robotic instrument 5400, which causes the second tool 5463 (e.g., cutting tool) to translate along the z-axis (i.e., proximally and distally). As described above, the second output drive member 5720 is coupled to actuation elements (e.g., cables) that are coupled to the end effector 5460 and second tool 5463 and can control movements of the second tool 5463 (e.g., and cutter element 5482) along the z-axis direction (proximally and distally).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

Any of the components of a surgical instrument described herein can be constructed from any material, such as medical grade stainless steel, nickel alloys, titanium alloys or the like. Further, any of the links, tool members, tension members, or components described herein can be constructed from multiple pieces that are later joined together. For example, in some embodiments, a link can be constructed by joining together separately constructed components. In other embodiments however, any of the links, tool members, tension members, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A manual drive adapter for a robotic instrument, comprising:
an adapter housing configured to be releasably coupled to a force transmission mechanism of the robotic instrument, wherein the force transmission mechanism of the robotic instrument comprises a robotic drive member interface and a robotic input drive member configured to engage with a robotic drive output of a teleoperated surgical system;
an adapter output drive interface comprising an adapter output drive member, wherein the adapter output drive interface is coupled to the adapter housing and is configured to be releasably coupled to the robotic drive member interface of the robotic instrument such that the adapter output drive member is engageable with the robotic input drive member of the robotic instrument; and
a manual actuator operably coupled to the adapter output drive member such that movement of the manual actuator by a user causes the adapter output drive member to operate one or more degrees of freedom of the robotic instrument.

2. The manual drive adapter of claim 1, wherein when the adapter output drive interface of the manual drive adapter is coupled to the robotic drive member interface of the robotic instrument, the robotic input drive member of the robotic instrument is prevented from being engaged with a robotic drive output of a teleoperated surgical system.

3. The manual drive adapter of claim 1, wherein:
the robotic instrument includes a shaft having a proximal end portion coupled to the robotic input drive member of the robotic instrument and a distal end portion coupled to an end effector, and
movement of the manual actuator by the user causes the adapter output drive member to move the robotic input drive member of the robotic instrument to actuate the end effector.

4. The manual drive adapter of claim 3, wherein:
the end effector comprises a gripping tool, movement of the manual actuator actuates the gripping tool when the adapter output drive member is coupled to the robotic input drive member of the robotic instrument.

5. The manual drive adapter of claim 3, wherein:
the end effector comprises a cutting tool, movement of the manual actuator actuates the cutting tool to move relative to jaws of the end effector when the adapter output drive member is coupled to the robotic input drive member of the robotic instrument.

6. The manual drive adapter of claim 1, wherein the adapter output drive interface of the manual drive adapter comprises a plurality of adapter output drive members configured to be engaged with a plurality of robotic input drive members of the robotic instrument.

7. The manual drive adapter of claim 6, wherein:
the robotic input drive member is a first robotic input drive member of the plurality of robotic input drive members of the robotic instrument, the manual actuator is a first manual actuator, and the adapter output drive member is a first adapter output drive member of the plurality of adapter output drive members of the manual drive adapter, the manual drive adapter further comprising:
a second manual actuator operably coupled to a second adapter output drive member of the plurality of adapter output drive members, wherein the second manual actuator can be manually actuated by the user to cause the second adapter output drive member to move a second input drive member of the plurality of input drive members of the robotic instrument when the adapter housing is coupled to the force transmission mechanism of the robotic instrument.

8. The manual drive adapter of claim 7, wherein:
the robotic instrument includes a shaft having a proximal end portion coupled to the force transmission mechanism of the robotic instrument and a distal end portion coupled to an end effector of the robotic instrument, the end effector comprising a gripping tool and a cutting tool;
the first manual actuator is configured to manually actuate the gripping tool when the first adapter output drive member is coupled to the first robotic input drive member of the robotic instrument; and
the second manual actuator is configured to manually actuate the cutting tool to move relative to jaws of the gripping tool when the second adapter output drive member is coupled to a second robotic input drive member of the robotic instrument.

9. The manual drive adapter of claim 7, wherein:
the robotic instrument includes a shaft having a proximal end portion coupled to the force transmission mechanism of the robotic instrument and a distal end portion coupled to a wrist mechanism of the robotic instrument, the wrist mechanism configured to rotate about a rotation axis to move a position of the wrist mechanism about the rotation axis;
the first manual actuator configured to manually actuate the wrist mechanism when the first adapter output drive member is coupled to the first robotic input drive member of the robotic instrument; and
a second manual actuator configured to cause the wrist mechanism to be locked in a fixed position relative to the shaft.

10. The manual drive adapter of claim 8, wherein:
the distal end portion of the shaft is coupled to a link, the link is coupled to the gripping tool and the cutting tool, the link configured to rotate about a rotation axis to move a position of at least one of the gripping tool or the cutting tool about the rotation axis, the manual drive adapter further comprising:
a third manual actuator configured to be manually actuated by the user to lock the link in a fixed position relative to the shaft and prevent at least one of the gripping tool or the cutting tool from moving about the rotation axis.

11. The manual drive adapter of claim 7, further comprising:
a lockout mechanism that prevents the second manual actuator from being actuated unless the first manual actuator is actuated.

12. The manual drive adapter of claim 1, further comprising:
a torque limiter coupled to the manual actuator and configured to limit a torque applied to the adapter output drive member when the manual actuator is moved by the user.

13. The manual drive adapter of claim 12, wherein the torque limiter comprises a torsion spring.

14. The manual drive adapter of claim 1, further comprising:
a power connection configured such that when the adapter housing is coupled to the force transmission mechanism of the robotic instrument, the robotic instrument is electrically coupled to a power source.

15. The manual drive adapter of claim 1, wherein the manual drive adapter is provided within a sterile kit and configured for a single use.

16. A manual drive adapter, comprising:
an adapter housing configured to be releasably coupled to a force transmission mechanism of a robotic instrument, the force transmission mechanism of the robotic instrument including a first robotic input drive member and a second robotic input drive member, the first robotic input drive member configured to engage with a first robotic drive output of a teleoperated surgical system, the second robotic input drive member configured to engage with a second robotic drive output of the teleoperated surgical system;
a first manual actuator coupled to the adapter housing and configured to be coupled to the first robotic input drive member such that movement of the first manual actuator causes the first robotic input drive member to operate a first tool of the robotic instrument; and
a second manual actuator coupled to the adapter housing and configured to be coupled to the second robotic input drive member such that movement of the second manual actuator causes the second robotic input drive member to operate a second tool of the robotic instrument.

17. The manual drive adapter of claim 16, wherein the robotic instrument includes a shaft having a proximal end portion coupled to the force transmission mechanism of the robotic instrument and a distal end portion coupled to a link, the link being coupled to the first tool and configured to rotate about a rotation axis to move a position of the first tool about the rotation axis, the manual drive adapter further comprising:
a third actuator configured to be manually actuated to lock the link in a fixed position relative to the shaft and prevent the first tool from moving about the rotation axis.

18. The manual drive adapter of claim 16, wherein:
the first tool is a gripping tool, movement of the first manual actuator actuates the gripping tool when the adapter housing is coupled to the force transmission mechanism of the robotic instrument, and
the second tool is a cutting tool, movement of the second manual actuator actuates the cutting tool to move relative to jaws of the gripping tool when the adapter housing is coupled to the force transmission mechanism of the robotic instrument.

19. The manual drive adapter of claim 16, further comprising:
a lockout mechanism configured to prevent the second manual actuator from being actuated unless the first manual actuator is actuated.

20. The manual drive adapter of claim 16, further comprising:
a torque limiter coupled to the first manual actuator and configured to limit a torque applied to the first robotic input drive member when the first manual actuator is moved.

21. The manual drive adapter of claim 16, further comprising:
a power connection configured such that when the adapter housing is coupled to the force transmission mechanism of the robotic instrument, the second tool is electrically coupled to a power source.

22. The manual drive adapter of claim 16, wherein the manual drive adapter is provided within a sterile kit and configured for a single use.
